SEQUENCE LISTING (13 sequences)

SEQ ID NO: 1 - DNA, 19 bp, Artificial Sequence
  DNA adaptor for ligation
gcagaatccgaggccgcct

SEQ ID NO: 2 - DNA, 24 bp, Artificial Sequence
  DNA adaptor for ligation
gacaaggcggcctcggattctggc

SEQ ID NO: 3 - DNA, 19 bp, Artificial Sequence
  DNA adaptor for ligation
agtggcgtgtcttggatgc

SEQ ID NO: 4 - DNA, 26 bp, Artificial Sequence
  DNA adaptor for ligation
cgataacgcatccaagacacgccact

SEQ ID NO: 5 - DNA, 22 bp, Artificial Sequence
  PCR primer combining adaptor and genomic sequences
gacaaggcggcctcggattctg

SEQ ID NO: 6 - DNA, 22 bp, Artificial Sequence
  PCR primer combining adaptor and genomic sequences
cgataacgcatccaagacacgc

SEQ ID NO: 7 - DNA, 22 bp, Artificial Sequence
  Labeled PCR primer
gacaaggcggcctcggattctg

SEQ ID NO: 8 - DNA, 22 bp, Artificial Sequence
  Labeled PCR primer
cgataacgcatccaagacacgc

SEQ ID NO: 9 - DNA, 44 bp, Artificial Sequence
  Labeled circularization linker
cgtcttacgcgccggcggaatccgtcttacgcgccggcggaatc

SEQ ID NO: 10 - DNA, 32 bp, Artificial Sequence
  Aminated oligonucleotide used for fixing circularized molecules to a slide
aaaaaaaaaagcgtgtcttggatgcgttatcg

SEQ ID NO: 11 - DNA, 22 bp, Artificial Sequence
  DNA Adaptor with internal NotI site
gcagaatgcgcggccgccttag

SEQ ID NO: 12 - DNA, 44 bp, Artificial Sequence; identical to SEQ ID NO: 9
  Linker oligonucleotide

SEQ ID NO: 13 - DNA, 54 bp, Artificial Sequence
  Primer used in immobilization
cgtcttacgcgccggcggaatccgtcttacgcgccggcggaatcaaaaaaaaaa

(19) United States
(12) Patent Application Publication
(10) Pub. No.: US 2010/0028873 A1
(43) Pub. Date: Feb. 4, 2010

(54) METHODS AND MEANS FOR NUCLEIC ACID SEQUENCING

(76) Inventors: Abdelmajid Belouchi, Quebec (CA); Steve Geoffroy, Montieal (CA); Sten Linnarsson, Stockholm (SE); Pierre Berube, Montieal (CA)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/293,013

(22) PCT Filed: Mar. 14, 2007
(86) PCT No.: PCT/US07/06372

§ 371 Date: Jun. 24, 2009

Related U.S. Application Data
(60) Provisional application No. 60/781,731, filed on Mar. 14, 2006.

(51) Int. Cl. C12Q 1/68

(52) U.S. Cl. 435/6

(57) ABSTRACT

The present invention provides a nucleic acid sequencing method. The method comprises enriching a nucleic acid sample for target nucleic acids, where the nucleic acid sample is enriched through at least a first round of hybridization selection and amplification, and a second round of hybridization selection and amplification. The enriched nucleic acids are in a form convenient for sequencing with the Cantaloupe sequencing technology, which employs shotgun sequencing by hybridization (SBH) of immobilized rolling circle amplicons.

This application claims the benefit of U.S. Provisional Application No. 60/781,731 filed Mar. 14, 2006, the entire disclosure of which is hereby incorporated by reference in its entirety.

The present invention relates to nucleic acid sequencing, and particularly to the sequencing methods disclosed in PCT/EP2005/002870 (corresponding to WO 2005/093094), the entire disclosure of which is hereby incorporated by reference in its entirety.

### BACKGROUND OF THE INVENTION

Although many different methods are used in genomic research, direct sequencing is by far the most valuable. In fact, if sequencing could be made efficient, then the three main facets of genomics analysis (sequence determination, genotyping, and gene expression analysis) could be addressed. For example, a model species could be sequenced, individuals could be genotyped by whole-genome sequencing, and RNA populations could be exhaustively analyzed after conversion to cDNA.

Other analyses that may be improved by advances in sequencing technology include: epigenomics (e.g., methylated cytosines could be identified by bisulfite conversion of unmethylated cytosine to uridine), identifying protein-protein interactions (e.g., by sequencing hits obtained in a yeast two-hybrid experiment), identifying protein-DNA interactions (e.g., by sequencing DNA fragments obtained after chromosome immunoprecipitation), and many others.

Thus, highly efficient methods for DNA sequencing are desirable. Specifically, high throughput sequencing methods are needed. For example, a living cell contains about 300,000 copies of messenger RNA, each about 2,000 bases long on average. To completely sequence the RNA in even a single cell, 600 million nucleotides must be analyzed. In a complex tissue composed of dozens of different cell types, the task becomes even more difficult as cell-type specific transcripts become diluted. Gigabase daily throughput will be required to meet these demands. The following table shows some estimates on the throughput required for various sequencing projects (numbers are for human sequencing, unless otherwise indicated):

| Experiment | Throughput required | |
|---|---|---|
| Genome sequence (10x de novo) | 30 | Gbp |
| Whole-genome polymorphisms | 3 | Gbp |
| Complete haplotype map (200 individuals) | 600 | Gbp |
| Gene expression | 600 | Mbp |
| Epigenomics | 3 | Gbp |
| Ten million protein interactions | 400 | Mbp |
| Entire biosphere (one species per genus) | ~300 | Tbp |

A number of different sequencing technologies have been developed.

Sanger sequencing (Sanger et al., PNAS 74 no. 12: 5463-5467, 1977) using fluorescent dideoxy nucleotides, is the most widely used method, and has been successfully automated in 96 and even 384-capillary sequencers. However, the Sanger method relies on the physical separation of a large number of fragments corresponding to each base position of the template and is thus not readily scalable to ultra-high throughput sequencing (the best current instruments generate ˜2 million nucleotides of sequence per day).

Sequencing-by-hybridization (SBH) uses a panel of probes representing all possible sequences up to a certain length (e.g., a set of all 10-mers requires over one million probes). However, for a given set of all “k-mers,” k will be limited by the number of probes that can fit on the microarray surface. Further, reconstructing the template sequence from the hybridization data is complicated, and made more difficult by the nature of hybridization kinetics and the combinatorial explosion of the number of probes required to sequence larger templates. The throughput is therefore low, as one microarray carrying millions of probes is required for each template.

An alternative approach to SBH is to place the template on the solid surface and then sequentially hybridize the panel of probes. Using this approach, many templates can be sequenced in parallel, but the size of the panel of probes is necessarily limited by the sequential nature of the protocol. As a consequence, only very short templates can be sequenced. In fact the expected length that can be sequenced with k-mer probes is only 2^{k}, or 128 nucleotides using 16384 probes (k=7). With realistic hybridization times, such a protocol is not feasible. Drmanac et al., Nature Biotech 16:54-8 (1998) attempt to address this problem by replicating each template on hundreds of separate membranes that may then be hybridized in parallel. However, this strategy limits throughput and places additional demands on the template preparation method.

In nanopore sequencing (e.g., U.S. Pat. No. 6,355,420) a DNA molecule is forced through a nanopore that separates two reaction chambers, which allows bound probes to be detected by changes in the conductance between the chambers. By decorating DNA with a subset of all possible k-mers, it is possible to deduce a partial sequence. So far, no viable strategy has been proposed for obtaining a full sequence by the nanopore approach, although if it were possible, staggering throughput could in principle be achieved (on the order of one human genome in thirty minutes).

Various approaches have been designed for sequencing by synthesis (SBS), which involve either detecting a byproduct released from incorporated nucleotides, or detecting a permanently attached label. To increase throughput for SBS it would be desirable to visualize the incorporation of each base on a large number of templates in parallel, e.g. on a glass surface or similar reaction chamber (see, e.g., U.S. Pat. No. 4,863,849 and U.S. Pat. No. 5,908,755).

For example, pyrosequencing (e.g., WO/9323564) determines the sequence of a template by detecting the byproduct of each incorporated monomer in the form of inorganic diphosphate (PPi). To keep the reactions of all template molecules synchronized, monomers are added one at a time and unincorporated monomers are degraded before the next addition. However, homopolymeric subsequences (runs of the same monomer) pose a problem as multiple incorporations cannot be prevented. Synchronization eventually breaks down due to misincorporation at a small fraction of the templates eventually overwhelming the true signal. The best available systems can read only about 20-30 bases with a combined throughput of about 200,000 bases/day.

U.S. Pat. No. 6,274,320 describes the use of rolling-circle amplification to produce tandemly repeated linear single-stranded DNA molecules attached to an optic fiber, which are analyzed in a pyrosequencing reaction that can then proceed in parallel. In principle, the throughput of such a system is limited only by the surface area (number of template molecules), the reaction speed and the imaging equipment (resolution). However, the need to prevent PPi from diffusing away from the detector before being converted to a detectable signal limits the number of reaction sites in practice. In U.S. Pat. No. 6,274,320, each reaction is constrained to occur in a miniature reaction vessel located on the tip of an optic fiber, thus limiting the number of sequences to one per fiber.

Even more limiting are the short read-lengths achieved by Pyrosequencing (<50 bp). Such short sequences are not always useful in whole-genome sequencing, and the complex set of balancing reactions make it difficult to extend the read-length much further. Only occasionally and for specific templates have read lengths up to 100 bp been reported.

A scheme detecting a released label is described in U.S. Pat. No. 6,255,083, and a scheme with sequential addition of nucleotides and detection of a label that is then removed with an exonuclease is described in WO01/23610. The principal advantage of detecting a released label or byproduct is that the template remains free of label at subsequent steps. However, because the signal diffuses away from the template, it may be difficult to parallelize such sequencing schemes on a solid surface such as a microarray.

Improved and more efficient sequencing methods, such as those with higher throughput, would in turn allow for more efficient and improved genomic analysis.

Searching for the genetic variants and mutations that underlie human diseases, both simple and complex, presents many challenges. In the case of complex diseases, these searches generally result in single nucleotide polymorphisms (SNP), or sets of SNPs, associated with disease risk. The identification of all genes and genetic traits associated with a complex disease, such as Crohn's disease, Psoriasis, Asthma and Schizophrenia, has not been possible to date. The main reason is that the methods available for genomic analysis are time-consuming and thus create bottlenecks in the process.

In whole-genome association studies (WGAS), many candidate regions (CRs) have been identified and such regions must be sequenced to identify the genes, genetic variants and polymorphisms associated with disease. However, CRs can be quite large (>100 kb), and thus sequencing many CRs in many individuals, presents a tremendous sequencing burden.

### SUMMARY OF THE INVENTION

The present invention relates to “high-density fingerprinting,” in which a panel of nucleic acid probes is annealed to nucleic acid for which sequence information is desired. By determining the presence or absence of sequence complementarity between each probe and target nucleic acids, sequence information is determined. The invention is based in part on using a reference sequence related to the template, which overcomes various problems with existing sequencing techniques, and allows for a large amount of sequence to be obtained in a short time using standard reagents and apparatus. Preferred embodiments provide additional advantages.

The invention also relates to algorithms and techniques for sequence analysis, and apparatus and systems for sequencing. The present invention allows for automation of a vast sequencing effort, using only standard bench-top equipment that is readily available in the art.

The invention involves hybridization of a panel of probes, each probe comprising one or more oligonucleotide molecules, in sequential steps, and determining for each probe if it hybridizes to the template or not, thus forming the “hybridization spectrum” of the target. Preferably, the panel of probes and the length of the template strand are adjusted to ensure dense coverage of any given template strand with “indicative probes” (probes which hybridize exactly once to the template strand). The invention further involves comparing the obtained hybridization spectrum with a reference database expected to contain one or more sequences similar to the template strand, and determining the likely location or locations of the template strand within one or more reference sequences. The invention further allows for the hybridization spectrum of the template strand to be compared to the expected hybridization spectrum at the location or locations, thereby obtaining at least partial sequence information of the template strand.

The invention further relates to the field of genomics and genetics, including genome analysis and the study of DNA variations. Specifically, the invention contemplates enrichment of a DNA sample for DNA segments of interest. The segments of interest may represent candidate regions (CRs) identified from whole genome association studies of disease, for example. CRs may be: genomic DNA sequences; intergenic DNA sequences; sequences that correspond to gene elements, such as promoters, exons, introns, UTRs, and conserved non-coding sequences; or cDNA sequences. The present invention is useful for, inter alia, identifying single nucleotide polymorphisms (SNPs), other types of polymorphisms (insertions, deletions, microsatellites), as well as specific alleles and haplotypes associated with disease. The methods of the invention provide for the discovery of DNA variation and polymorphisms in the fields of pharmacogenomics, diagnostics, patient therapeutics and the use of genetic haplotype information to predict an individual's susceptibility to disease or complex genetic trait and/or their response to a particular drug or drugs, so that drugs tailored to genetic differences of population groups may be developed and/or administered to the appropriate population.

The invention provides methods for selection and sequencing CRs at a fast, accurate and cost-effective rate. Specifically, the invention couples a DNA fragment enrichment technology to a sequencing technology named “Cantaloupe” (described in detail in WO2005/093094, which is herein incorporated by reference in its entirety). The Cantaloupe technology enables the sequencing of an entire human genome in about 10 days. While enrichment technologies have been described (see, e.g., Lovett et al., PNAS 88:9628-9632, 2005; and Bashiardes et al., Nat. Methods 2(1): 63-69, 2005, which are herein incorporated by reference in their entireties), the present invention provides an enrichment method that produces DNA fragments compatible with the Cantaloupe sequencing platform.

Using the method of the invention, genomic DNA fragments are enriched for sequences of interest, which may then be conveniently and easily sequenced by the Cantaloupe technology, thus permitting high-throughput sequencing of large DNA fragments in a time- and cost-effective manner. In an exemplary embodiment, DNA, such as genomic DNA, is fragmented and fragments of a desired size are selected. DNA adapters containing primer binding sites, as describe more fully herein, are ligated to the fragments. At least two rounds of hybridization selection with a nucleic acid probe and amplification produce an enriched sample. Single-stranded fragments of the enriched sample are then produced and circularized, and immobilized to a solid support. The immobilized DNA is then replicated by rolling circle amplification (RCA) mechanism, to form a random array of rolling circle (RC) amplification products. A series of probes are sequentially hybridized to the RC products to produce a hybridization spectrum. The probes consist (for example) of 7-mer oligonucleotides; with 5 variable bases and 2 fixed bases, for a total of 1,024 possible different probes. The hybridization spectra is like a bar code for each fragment, which may then be compared to a reference sequence. The sequence of the target nucleic acid is then reconstructed by “assembling” and comparing all the fragment bar codes to a reference genome.

Thus, the present invention has the capability to select for regions of interest, from, for example, a sample of genomic DNA, and to produce genetic material in a form that is ready for automated sequencing systems, such as the Cantaloupe technology. The method of the invention results in the rapid, efficient and cost-effective analysis and identification of DNA variations.

### BRIEF DESCRIPTION OF THE FIGURES

FIG. 1 shows a gel image which shows the result of cleaving a cDNA sample (lane 4) with CviJ* for increasing durations. A gradual reduction in the average fragment length towards 100 bp is observed (100 bp is the lowest fragment of the size standard, lane 3). The optimal cleavage reaction is loaded in lane 1 and fragments around 100 bp are purified.

FIG. 2 shows adapter ligation. Lane 1 is the size marker; lane 2, unligated fragments; lanes 3 and 4, ligated fragments. Most fragments are correctly ligated.

FIG. 3 Shows the sample of fragments before (lane 1) and after (lane 2) circularization. Lane 3 shows the result after purification. Notice the absence of linker in lane 3.

FIG. 4 shows a section of approximately 0.8 by 2.4 mm from a random array slide scanned using a Tecan™ LS400 at 4 μm resolution using the 488 nm laser and 6FAM filter. Spots represent amplification products generated from individual circular template molecules.

FIG. 5 shows the stability of short oligonucleotide probes measured by melting point analysis. FIG. 5A shows the effect of CTAB in 100 mM tris pH 8.0, 50 mM NaCl. FIG. 5B shows the effect of LNA in TaqExpress buffer (GENETIX, UK). FIG. 5C shows the specificity of LNA in TaqExpress buffer. FIG. 5D shows the effect of introducing degenerate position: 7-mer with 5 LNA (left), 7-mer with 5 LNA and 2 degenerate positions (middle), 7-mer with 3 LNA and 2 degenerate positions (right).

FIG. 6 shows a FAM-labeled universal 20-mer probe (left panel) and a TAMRA-labeled 7-mer probe (middle), hybridized to a random array and visualized by fluorescence microscopy. The array was synthesized with two templates, both of which should bind the universal probe but only one of which should bind the 7-mer at the sequence CGAACCT. The image was captured using a Nikon DS1QM COD camera at 20× magnification on a Nikon TE2000 inverted microscope. The right-hand panel shows a color composite, and demonstrates that all TAMRA-labeled features were also FAM-positive, as expected.

FIG. 7 shows steps for enriching a DNA sample for target sequences of interest for sequencing by Cantaloupe.

### DETAILED DESCRIPTION OF THE INVENTION

### Enriching for Target Sequences

The present invention encompasses a method for enriching a nucleic acid sample for target sequences of interest, for subsequent sequencing by hybridization (SBH) of immobilized rolling circle amplicons. The method of the invention comprises a first round of hybridization selection and amplification, and a second round of hybridization selection and amplification. Additional rounds of selection and amplification may be employed for further enrichment of the nucleic acid sample.

Any nucleic acid sample may be used in accordance with the invention, such as genomic DNA, cDNA, or RNA.

Target nucleic acids of interest may be nucleic acid segments identified from whole genome association studies in a disease cohort. Such a disease cohort may comprise DNA samples from patients with diseases or complex genetic traits such as: Crohn disease, psoriasis, baldness, longevity, schizophrenia, diabetes, diabetic Retinopathy, ADHD, Endometriosis, asthma, an autoimmune related diseases, an inflammatory related diseases, a respiratory related diseases, a gastrointestinal related diseases, a reproduction related disease, a women's health related diseases, a dermatological related diseases, and an ophthalmologic related disease.

The nucleic acid sample, such as a DNA sample, may be prepared for enrichment by fragmenting the DNA sample to create a population of DNA fragments, and ligating DNA adaptors to the DNA fragments. In one embodiment, the DNA adaptors contain primer binding sites to facilitate amplification after hybridization selection. Various fragmentation procedures are known in the art, but in one embodiment, the DNA sample is fractionated using DNase I and Mung Bean nuclease to create blunt-ended DNA fragments, such that the DNA adaptors, also having a blunt end, may be blunt-end ligated to the DNA fragments.

In one embodiment, DNA fragments of about 500 base pairs or smaller are selected for ligation to the DNA adapters, and in another embodiment, DNA fragments of about 200 or about 250 base pairs or smaller are selected.

In an exemplary embodiment of the invention, the first and second rounds of hybridization selection involve hybridizing the DNA sample, which may be fragmented and ligated to DNA adaptors as described above, with a nucleic acid probe having a tag. In one embodiment, the nucleic acid probe is a biotinylated bacterial artificial chromosome (BAC). The hybridized DNA may then be physically captured, for example, with streptavidin coated beads. Thus, in an exemplary embodiment, the tag and ligand are biotin and streptavidin, respectively. The streptavidin may be contained on particles or beads, for instance magnetic beads, to facilitate separation of the captured hybridized complexes. Numerous other equivalent tags are known in the art, and which may be used in conjunction with the present invention.

The nucleic acids of interest selected by the first round of hybridization selection are subsequently amplified in the first round of amplification. The first round of amplification may be performed using polymerase chain reaction (PCR), for example, but may be performed using any amplification procedure known in the art. The amplification is most readily performed using primers complementary to the DNA adapters, which, as described above, may be ligated to the DNA fragments.

The amplified nucleic acids of interest from the first round of amplification are further enriched in a second round of hybridization selection, using the techniques described briefly above and in more detail below. The nucleic acids of interest selected by the second round of hybridization selection are then subsequently amplified in the second round of amplification. The second round of amplification may also be performed using polymerase chain reaction (PCR), but likewise may be performed using any amplification procedure known in the art. The second amplification is also most readily performed using primers complementary to the DNA adapters, which are ligated to the nucleic acid fragment in the exemplary embodiment described above.

In the second round of amplification, or the final round of amplification as the case may be, the primers may be modified to facilitate further preparation for sequencing by hybridization. For instance, a second round amplification primer, e.g. the forward primer, may be modified on the 5′ end with a tag, for example, a biotin tag, and the other primer (e.g., the reverse primer) may be phosphorylated at the 5′ end. In accordance with this embodiment, the second round amplification products may then be denatured to create single-stranded nucleic acids, whereupon the tagged strands may be captured and removed using a ligand for the tag (e.g, streptavidin). The phosphorylated strands of the single stranded amplification products are then circularized. In one embodiment, the phosphorylated strands are circularized by hybridizing the 5′ and 3′ ends to an oligonucleotide linker, thereby holding the 5′ and 3′ ends in close proximity; and ligating the 5′ and 3′ ends to circularize the single-stranded DNA. A gap-fill polymerization step may be used to fill in any gap between the two ends prior to ligation. The oligonucleotide linker used to facilitate circularization may also be tagged, for example, with biotin, to facilitate its removal following circularization.

The circularized single stranded molecules may then be immobilized on a solid support. The nucleic acids of interest may be immobilized using any method known in the art, for instance using an aminated oligonucleotide as described herein. The immobilized, circularized nucleic acids of interest are then amplified using rolling circle amplification and sequenced using SBH, as described in WO 2005093094, which is herein incorporated by reference in its entirety.

The average candidate region size, based on genome wide association studies in diseases or complex genetic traits, such as Crohn's and psoriasis, is about half a megabase (0.5 Mb). In one embodiment of the invention, all candidate regions associated with a disease are selected. In another embodiment, only some candidate regions are selected. In yet another embodiment, a single candidate region, or a portion or portions of a candidate region, associated with the disease are selected for analysis.

Once a region or regions are selected for sequencing, a nucleic acid probe(s) can be selected or designed. Generally, the nucleic acid probe is a specific DNA molecule that covers an entire chromosomal region, such as a candidate region resulting from WGAS studies. The probe can also cover part of a candidate region. Suitable probes include YACs, BACs, cosmids, or phages. In one embodiment, nucleic acid probes are selected from BAC molecules available commercially and are specific to the candidate regions of interest. In another embodiment, BAC molecules are selected from non-commercial sources or are created from specific individuals of interest.

The nucleic acid probe may be prepared using common molecular biology techniques known in the art. For example, the BAC-DNA may be isolated and purified by well known methods, such as using the QIAGEN® Large-Construct Kit (as described by the manufacturer).

DNA samples may be selected from individuals affected by a particular disease (disease samples), or from unaffected individuals, which in one embodiment may be used as a control (control samples). For example, from 1 to 50 samples may be selected from affected individuals (disease samples), or in another embodiment, more than 50 samples are selected from affected individuals. Disease samples represent specific combinations of haplotypes, including risk, neutral, protective and rare haplotypes, covering all candidate regions of interest. In yet another embodiment, from 1 to 50 samples from healthy individuals are selected as controls, or more than 50 samples from healthy individuals are selected as controls.

The genomic DNA may be isolated and prepared by any known method in the art. The quality of the genomic DNA can be assessed by gel electrophoresis and the DNA concentration can be determined by standard methods, such as the picogreen dye DNA quantification method.

After the standard preparation and purification of the genomic DNA, the genomic DNA samples may be treated consecutively by two enzymatic steps to generate blunt-ended DNA fragments. In one embodiment, the DNA fragments are about 250 base pairs. In another embodiment, the fragments are smaller than 250 base pairs, i.e. about 25 bp, about 50 bp, about 100 bp, about 150 bp, about 200 bp, etc. In another embodiment, the fragments are longer than 250 base pairs, i.e., about 300 bp, about 350 bp, about 400 bp, about 450 bp, about 500 bp, about 1000 bp, or more. A preferred target fragment size of the present invention ranges from about 200 bp to about 400 bp. The enzymatic reactions of the invention are not limited to any particular enzymatic reaction. In one embodiment, the enzymes are DnaseI and Mung Bean nuclease I. In another embodiment, other non-enzymatic fractionation methods, such as sonication or shearing, may be used, as described further herein. Preferably, the fragmentation method results in blunt-ended fragments.

The resulting blunt-ended fragments are then ligated to DNA adaptors. In one embodiment, the blunt-ended fragment are ligated to the following DNA adaptors:

### Adaptor-1

| | |
|---|---|
| 5′-GCAGAATCCGAGGCCGCCT-3′ | (SEQ ID NO: 1) |
| 3′-CGTCTTAGGCTCCGGCGGAACAG-5′ | (SEQ ID NO: 2) |

### Adaptor-2

| | |
|---|---|
| 5′-AGTGGCGTGTCTTGGATGC-3′ | (SEQ ID NO: 3) |
| 3′-TCACCGCACAGAACCTACGCAATAGC-5′ | (SEQ ID NO: 4) |

The DNA adaptors are designed to only permit ligation at one end, and on the blunt-end part of the genomic DNA fragments. The ligation reaction can be performed by any method, and many are known in the art. In one embodiment, the adapters are added in excess in relation to the genomic DNA fragments.

The fragments ligated to the adaptors (genomic-adaptor DNA) are then separated and purified by any separation and purification method, of which many are known in the art, such as by electrophoresis on 12% non-denaturing polyacrylamide gels or 3.5% Metaphor agarose gels (Cambrex, Baltimore, Md.). Preferably, the fragments of interest are separated by electrophoresis, eluted, purified (GFX column GE Healtcare) from the gel, and quantified by any DNA quantification method, such as picogreen dye DNA quantification.

In another embodiment, the genomic-adaptor DNAs are purified from repetitive sequences. This purification is generally carried out by a hybridization reaction with competitive DNA, such as biotinylated Cot_{1/2} (Invitrogen). In yet another embodiment, any known purification method to remove repetitive sequences can be used. The resulting purified genomic-adaptor DNA may be used as in input genomic DNA for the first enrichment step of the present invention.

When BAC DNA is used as the nucleic acid probe, the BAC DNA may be tagged or labeled by the addition of biotin molecules to fragmented BAC-DNA, to provide a means for easy separation from other reaction components. In this embodiment, the BAC-DNA may be captured with streptavidin-coated magnetic beads, for example. Methods of tagging or labeling the BAC DNA are known in the art, such as with a Biotin-Nick Translation Mix. The nick translation method utilizes a combination of DNase and E.coli DNA Polymerase I to nick one strand of the DNA, and then incorporate labeled nucleotides as the polymerase re-synthesizes from the nicked site. Equivalent methods of labeling the BAC DNA are known in the art, and may be used in conjunction with this embodiment.

As for the genomic DNA preparation, BAC-DNA repeats on the probe are preferably blocked with competitive DNA, such as Cot-1 DNA (Invitrogen). In another embodiment, any other known method can be used for blocking the repeated sequences on the BAC-DNA probe.

The methods of the invention comprise at least one, but preferably at least two rounds of enrichment. Briefly, the first round enriches targeted DNA fragments from whole genomic DNA, while the second round enriches for targeted DNA fragments from the first round by reducing the amount of contaminating fragments. In both enrichment steps, the preferred end products are DNA fragments of ˜250 bp. In an other embodiment, such fragments can be smaller than 250 bp, i.e. from about 25 bp to about 250 bp, from about 50 bp to about 250 bp, from about 100 bp to about 250 bp, from about 150 bp to about 250 bp, or from about 200 bp to about 250 bp. In another embodiment, the fragments can be longer than 250 bp, i.e., about 300 bp or more, about 350 bp or more, about 400 bp or more, about 450 bp or more, about 500 bp or more, about 1000 bp or more, etc.

The preferred level of enrichment for the present invention is at least 1000 fold. However, enrichment levels of at least 200 fold or more, at least 500 fold or more, at least 1500 fold or more, at least 2500 fold or more, at least 5000 fold or more, etc., are also contemplated. The DNA fragments of the present invention, after enrichment, have the features necessary for the Cantaloupe sequencing technology.

In one embodiment, each enrichment step comprises a hybridization between the nucleic acid probe and the nucleic acid sample (e.g., fragmented genomic DNA with adaptors), binding of the hybridization product to a solid media (such as streptavidin-coated magnetic beads), amplification of the selected nucleic acids, and a nucleic acid cleanup step.

In one embodiment, hybridization between the nucleic acid probe and the DNA sample (e.g., adaptor-genomic DNA as described above) involves a hybridization reaction between purified adaptor-genomic DNA and blocked BAC-DNA. The hybridization mixture is then hybridized to any solid media capable of recognizing and binding the hybridization mixture. Preferably, such solid media comprises additional features that make the isolation of such hybridization complex easy. In one embodiment, such solid media is streptavidin-coated magnetic beads. Hybridization reactions are well known in the art and the present invention does not limit itself to any particular conditions for hybridization. Exemplary conditions are shown in Example 1 herein. The DNA collected from the solid media is purified and concentrated for use in a subsequent PCR amplification reaction. Other known amplification procedures may also be used, for instance NASBA, SDA, etc.

The first PCR amplification step of the present invention is performed using 2 primers (one forward and one reverse), each containing an adaptor sequence ligated to the genomic DNA fragments. In an exemplary embodiment, the primer sequences are:

(SEQ ID NO: 5)

Forward: 5′-GACAAGGCGGCCTCGGATTCTG-3′

(SEQ ID NO: 6)

Reverse: 5′-CGATAACGCATCCAAGACACGC-3′

PCR amplification reagents are well described in the art and contain nucleotides, enzymes and buffers. The cycling parameters usually contain an initial denaturing step, followed by 25-30 cycles, each having a denaturing, an annealing and an elongation step. The amplification products are purified using any DNA purification method or kit, such as QIAquick PCR purification kits (QIAGEN) and are kept as input DNA for the second enrichment step.

The second enrichment of the present invention is performed as described in the first enrichment step with the input DNA being the amplification products from the first enrichment. The second amplification is similar to the first amplification described in the first enrichment above. However, in the second or final enrichment step, the primers may be modified to facilitate the preparation and circularization of single stranded DNA for sequencing by Cantaloupe. For example, the primers may be identical in base sequence to the primers used in previous enrichment steps, but that one primer may include a tag on its 5′-end, such as a biotin tag, and the other may have a 5′ phosphate. For example, the forward primer may have a 5′ biotin, and the reverse primer may have a 5′ phosphate, as shown:

(SEQ ID NO: 7)

Forward: 5′-BIOTIN-GACAAGGCGGCCTCGGATTCTG-3′

(SEQ ID NO: 8)

Reverse: 5′-PHO-CGATAACGCATCCAAGACACGC-3′

These modifications (biotinylation and phosphorylation) in the primers ensure that the resulting DNA fragments are ready for single stranded DNA preparation and circularization, to allow for convenient sequencing by the Cantaloupe sequencing technology. Specifically, streptavidin containing solid media may be used to capture and remove the biotinylated strand from the sample, so that the complementary strand (containing the phosphate group on the 5′-end) is purified and isolated. The single stranded linear fragments produced (with a phosphate group present on the 5′-end) may then be incubated with a linker capable of hybridizing to the 5′ and 3′ ends of the molecule, thus bringing the two ends in close proximity. An enzymatic reaction, such as ligation with DNA ligase, joins the 5′ and 3′ ends. A polymerization gap-fill reaction may also be used to fill in any gaps between the two ends prior to ligation. In an exemplary embodiment, the linker to aid in circularization is:

(SEQ ID NO: 9)

5′-BIOTIN-CGTCTTACGCGCCGGCGGAATCCGTCTTACGCGCCGGCGG
AATC-3′

As shown above, this linker may also contain a label or tag to facilitate its removal from the sample of circularized molecules.

The circularized single stranded DNA molecules are then immobilized for rolling circle amplification. In an exemplary embodiment, Asper Biotech Genorama™ SAL, 0.15 or 1 mm slides are used (in accordance with the manufacturer's instructions for handling and storage) for immobilizing the purified circular molecules. In another embodiment, any slide available commercially can be used to immobilize the circular molecules.

In one embodiment, an aminated oligonucleotide (see Diagram A below) is used to fix the circularized molecules to the slide. For example, the following exemplary oligonucleotide may be used:

(SEQ ID NO: 10)

5′ XAAAAAAAAAAGCGTGTCTTGGATGC♯GTTATCG ♭3′ RCA-G-RING
X═NH2—(CH2)6-PO4-Oligo

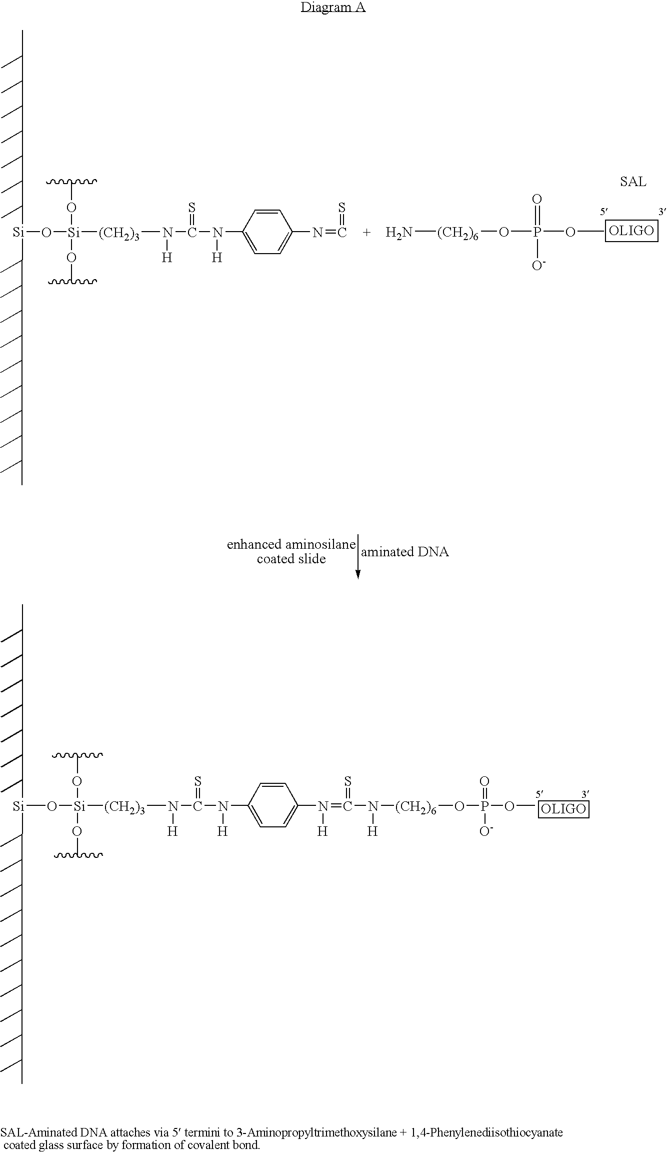

The present invention uses the nucleic acid sequencing technology described fully in patent application WO2005/093094 and incorporated here by reference, as the method to sequence the candidate regions enriched by the method described herein.

In the preferred embodiment, all candidate regions processed by the enrichment method described herein and immobilized on the glass slides are processed by the Cantaloupe sequencing technology.

### DNA Sequencing

As described above, circular single-stranded DNA template molecules are prepared for sequencing. Each of these template molecule comprises a primer annealing sequence and a target sequence, for which sequence information is desired. For sequencing, a random array of immobilized, circular DNA template molecules is formed, followed by rolling circle amplification using an amplification primer that anneals to the primer annealing sequence. The rolling circle amplification products are then hybridized with a panel of probes under test conditions to determine, for each probe in the panel, whether the probe hybridizes to the target sequence of the rolling circle amplification product, or not, thereby obtaining a hybridization spectrum for the target sequence. The hybridization spectrum may then be compared to an expected hybridization spectrum for a reference sequence(s) in a reference database, to determine the sequence of the target nucleic acid.

Amplifying the circular single stranded template molecules by rolling-circle amplification may comprise adding polymerase and triphosphates under conditions which cause elongation of the amplification primer and strand displacement to form a tandem-repeated amplification product comprising multiple copies of the target sequence.

The panel of probes employed may be a full panel or a partial panel as explained further below.

The reference sequence will be a similar sequence to target. Similarity between a reference sequence and a target can be measured in many ways. For example, the proportion of identical nucleotide positions is commonly used. More advanced measures allow for insertions and deletions e.g. as in Smith-Waterman alignment and provide a probabilistic similarity score as in Durbin et al. “Biological Sequence Analysis” (Cambridge University Press 1998).

The degree of similarity required for the method of the present invention is determined by several factors, including the number and specificity of the probes used, the quality of the hybridization data, the template length and the size of the reference database. For example, simulations show that under the assumption of degree melting point difference between match and mismatch probes (with 1 degree coefficient of variation), 256 probes and using the human genome as reference with 100 bp templates, then up to 5% sequence divergence can be tolerated. This corresponds for example to sequencing the Gorilla genome using the human genome as reference. Further increasing the number of probes, decreasing the length of the templates or improving the match/mismatch discrimination allows sequences of even lower similarity to be used as reference, e.g. 5-10%, up to 10%, 5-20%, 10-20% or up to 20%.

The present invention is applicable in various ways, including in resequencing, expression profiling, analysis or assessment of genetic variability, and epigenomics.

Various embodiments may be performed as follows.

A sample is fragmented to create a shotgun library of short fragments. The fragmentation methods described in the previous section may be used, especially where enrichment of sequences is desirable. Other enzymatic and/or mechanical methods of generating fragments may be employed, for example including:

Enzymatic:
Degradation with DnaseI (in the presence of Mn²⁺), then fill-in and/or enzymatic shortening of dangling ssDNA ends;
Cutting with a moderately frequent cutter, such as MboI etc.;
Partial cutting with a very frequent cutter, such as CviJI, CviJI* etc.;
Cutting with a mix of restriction enzymes;

Mechanical:
French press;
Sonication;
Shearing;
each of which may be followed by enzymatic shortening and end-repair;

PCR
using random priming sequences such as hexamers (optionally tailed with sequences for nested PCR);
by PCR using degenerate primers or low-stringency conditions;
by PCR using gene family-specific primers (etc.).

With PCR techniques, this step may optionally incorporate primer-binding sites, such as RCA (rolling circle amplification) primer annealing site or adaptors for enrichment.

Optionally following the fragmentation step a step “X” may be performed as described further below.

An RCA primer annealing sequence is added to the fragments. This may be for example, by cloning the fragments into a vector (e.g. bacterial vector, phage etc.), then excising the fragments using restriction enzymes placed outside the cloning site as well as the primer motif; or by ligation of double-stranded adaptors at one or both ends; or by ligation of hairpin adaptors at each end, which also provides simultaneous circularization. Optional additional, functional features that may be incorporated include features helping circularization and/or a helper oligo binding site, where a helper oligo can serve as donor or acceptor in FRET in downstream analyses.

Optionally a step “X” may be performed as described further below.

A sequencing method involves generating single-stranded circular DNA. This may be for example by ligation of hairpin adaptor after melting and self-annealing end-to-end in a maracas shape; by self-ligation of dsDNA followed by melting; by ligation to a helper fragment to form a dsDNA circle, followed by melting; by ligation of hairpin adaptors to both ends of dsDNA in a dumbbell shape; or by self-ligation of ssDNA using helper linker (which may also serve as an RCA primer).

Rolling circle amplification (RCA) may be performed in accordance with the following protocol:
Anneal an RCA primer to the circular ssDNA. The primer should carry a reactive moiety which can be used for immobilization.
Randomly immobilize the primer/template complex to the surface of an activated array using the attachment group of the RCA primer. The density of the primer/template complex on the surface should be optimized to allow for a maximum number of primer/template complexes on the surface without creating overlapping products after the RCA amplification (see below). The density of the primer/template complex on the surface may be controlled for example by the concentration of the primer/template complex, by the density of attachment sites on the surface and/or by the reaction conditions (time, buffer, temperature etc.). or
Randomly immobilize the primer to the surface of an activated array using the attachment group of the RCA primer. The density of the primer on the surface should be optimized to allow for a maximum number of primer/template complexes on the surface without creating overlapping products after the RCA amplification (see below). The density of the primer on the surface may be controlled for example by the concentration of the primer, by the density of attachment sites on the surface and/or by the reaction conditions (time, buffer, temperature etc.).
Anneal an RCA primer to the circular ssDNA. The primer should carry a reactive moiety which can be used for immobilization.

After immobilisation and annealing:
then
Add polymerase and the four dNTPs to initiate the rolling circle amplification.
Optionally incorporate fluorescent label in RCA which may serve as fluorescence donor or acceptor in FRET.
Optionally incorporate affinity tag in RCA which may be used for multiple purposes:
   For condensation of the RCA product by internal cross-linking using a multivalent linker molecule with affinity for the tag;
   For post-amplification labelling using a fluorescent label conjugated with a molecule with affinity for the tag.

Alternatively, RCA may be performed in solution and the product may be immobilized after amplification. For example, the same primer may be used for amplification and for immobilization. In another option, a modified dNTP carrying an immobilization group may be incorporated during amplification and the amplified product may then be immobilized using the incorporated immobilization group. For example, biotin-dUTP, or aminoallyl-dUTP (Sigma) may be used.

Sequence may then be determined. For example, in one embodiment, the full or partial sequence of the various templates on the array is determined using sequential hybridization of a panel of non-unique probes as described further below. The sequence information for each template may then be compared with a database of sequences representative of the sample under investigation thereby determining the relative proportion of each target within the sample and/or determining any genetic or other structural differences with respect to the database.

Step X, mentioned above, it is a step of selection of fragment size range (ideally with very good resolution—1-10% CV). Techniques that may be used include the following:
By gel electrophoresis and elution using
   PAGE with dsDNA
   PAGE with ssDNA
   Agarose gel;
By chromatography (e.g. HPLC, FPLC);
Using an affinity tag, e.g. a 3′-biotin on cDNA.

These steps provide disclosure of preferred and optional steps and ways of performing steps of a method in accordance with aspects and embodiments of the present invention. All combinations of disclosed features within the steps are provided herein as aspects and embodiments of the present invention as if set forth word-for-word herein.

Sequencing in accordance with the present invention may comprise three fundamental steps. First, a random array of locally amplified template molecules is generated (preferably in a single step) from a sample containing a plurality of template strands. Second, the random array is subjected to sequential hybridization with a panel of probes with determination of the presence or absence of sequences complementary to each probe in each amplified template on the array. Third, the hybridization spectrum thus obtained is compared to a reference sequence database with a method that allows the determination of likely insertions, deletions, polymorphisms, splice variants or other sequence features of interest. The comparison step may be further separated in a search step followed by an alignment step.

### Random Array Synthesis

There are many approaches to providing amplified templates at high density. First, amplified templates may be arrayed by mechanical means, which however requires separate amplification reactions for each individual template molecule (thus limiting throughput and increasing cost). Second, templates may be amplified in situ using in-gel PCR (e.g. as described in U.S. Pat. No. 6,485,944 and Mitra R D, Church G M, “In situ localized amplification and contact replication of many individual DNA molecules”, Nucleic Acids Research 1999: 27(24):e34), which however requires the use of a gel (thus severely interfering with subsequent hybridization reactions).

The present invention advantageously uses rolling-circle amplification to synthesize random arrays in a single reaction from a sample containing a plurality of template molecules. Densities up to 10⁵-10⁷ per mm² are achievable. A random array synthesis protocol employed in embodiments of the present invention may comprise:

a. Provide a surface (e.g. glass) with an activated surface.

b. Attach primers, preferably via a covalent bond, or, instead of a covalent bond, a strong non-covalent bond (such as biotin/streptavidin) may be used.

b. Add circular single-stranded templates, preferably at a density suitable for the detection equipment.

c. Anneal the templates to the primers.

d. Amplify using rolling-circle amplification to produce a long single-stranded tandem-repeated template attached to the surface at each position.

(see, e.g., Lizardi et al. describe “Mutation detection and single-molecule counting using isothermal rolling circle amplification”: Nature Genetics vol 19, p. 225).

Modifications to this procedure include preannealing the circular template molecules to activated primers before immobilization, and/or providing “open-circle” template molecules which are circularized upon annealing to the primer and closed using a ligation reaction.

A “suitable density” is preferably one that maximizes throughput, e.g. a limiting dilution that ensures that as many as possible of the detectors (or pixels in a detector) detect a single template molecule. On any regular array, a perfect limiting dilution will make 37% of all positions hold a single template (because of the form of the Poisson distribution); the rest will hold none or more than one.

For example, on a Tecan LS400 with a 6 μm pixel size, the 7.5×2.2 cm reaction surface holds 45 million pixels. With a limiting dilution (Poisson distribution), 37% of those would hold a single template, i.e. 17 million templates. Sequencing 150 nucleotides on each template yields 2.5 Gb of sequence in 150 cycles. With a cycle time of 5 minutes, daily throughput is about 5 Gbp, equivalent to two full sequences of the human genome. In practice, more than one pixel may be needed to reliably detect a feature, but the same reasoning holds whether the detector is a single pixel or multiple pixels.

Templates suitable for solid-phase RCA should optimize the yield (in terms of number of copies of the template sequence), while providing sequences appropriate for downstream applications. In general, small templates are preferable. In particular, templates can consist of a 20-25 bp primer binding sequence and a 40-500 bp insert, which may be a 40-150 bp insert. However, templates up to 500 bp or up to 1000 bp or up to 5000 bp are also possible, but will yield lower copy numbers and hence lower signals in the sequencing stage. The primer binding sequence may be used both to circularize an initially linear template and to initiate RCA after circularization, or the template may contain a separate RCA primer binding site.

In order to increase the signal generated from rolling circle-amplified templates it may be necessary to condense them. Since an RCA product is essentially a single-stranded DNA molecule consisting of as many as 1000 or even 10000 tandem replicas of the original circular template, the molecule will be very long. For example, a 100 bp template amplified 1000 times using RCA would be on the order of 30 μm, and would thus spread its signal across several different pixels (assuming 5 μm pixel resolution). Using lower-resolution instruments may not be helpful, since the thin ssDNA product occupies only a very small portion of the area of a 30 μm pixel and may therefore not be detectable. Thus, it is desirable to be able to condense the signal into a smaller area.

The RCA product may be condensed by using epitope-labeled nucleotides and a multivalent antibody as crosslinker. Alternative approaches include biotinylated nucleotides cross-linked by streptavidin.

Alternatively, condensation may be achieved using DNA condensing agents such as CTAB (see e.g. Bloomfeld ‘DNA condensation, by nultivalent cations’ in ‘Biopolymers: Nucleic Acid Sciences’).

In order to immobilise the RCA primer oligonucleotides to a surface, many different approaches have been described (see e.g., Lindroos et al. “Minisequencing on oligonucleotide arrays: comparison of immobilisation chemistries”, Nucleic Acids Research 2001: 29(13) e69). For example, biotinylated oligos may be attached to streptavidin-coated arrays; NH₂— modified oligos may be covalently attached to epoxy silane-derivatized or isothiocyanate-coated glass slides, succinylated oligos may be coupled to aminophenyl- or aminopropyl-derived glass by peptide bonds, and disulfide-modified oligos may be immobilised on mercaptosilanised glass by a thioudisulfide exchange reaction. Many more have been described in the literature.

### Resequencing by Sequential Hybridization of Short Probes

The sequencing approach of the present invention comprises hybridization of a panel of probes, with match/mismatch discrimination for each probe and target. The result is a “spectrum” of each target. Furthermore, a reference sequence is provided in which the spectrum is located and aligned so that differences in the sequence of the target with respect to the reference can be determined with high accuracy.

The panel of probes and the target length are optimized so that the spectra can be used both (1) to locate unambiguously each target sequence in the reference sequence and (2) to resolve accurately any sequence difference between the target and the reference sequence.

In order to fulfill the first requirement, the panel contains enough information (in the information-theoretic sense) to unambiguously locate the target. A single, long, specific probe is sufficient to locate a single specific target, but cannot be used since that would require separate probes for each possible target. Instead, short non-unique probes are used. An optimal panel would use probes with a 50% statistical probability of hybridizing to each target, corresponding to 1 bit of information per probe. 50 such probes would be capable of discriminating more that 1000 billion targets. Such panels have the additional advantage of being resilient to error and to genetic polymorphisms. Our experiments have shown that a panel of 100 4-mer probes is capable of uniquely placing 100 bp targets in the human transcriptome even in the presence of up to 10 SNPs.

In order to fulfill the second requirement, the panel of probes must cover the target and must be designed such that sequence differences result in unambiguous changes in the spectrum. For example, a panel of all possible 4-mer probes would completely cover any given target with four-fold redundancy. Any single-nucleotide change would result in the loss of hybridization of four probes and the gain of four other characteristic probes.

The sensitivity of a probe panel can be calculated:

A probe is a mixture of one or more oligonucleotides. The mixture and the sequence of each oligonucleotide defines the specificity of the probe. The dilution factor of a probe is the number of oligonucleotides it contains. The effective specificity of a probe is given by the length of a non-degenerate oligonucleotide with the same probability of binding to a target. For example, a 6-mer probe consisting of four oligonucleotides where the first position is varied among all four nucleotides (i.e. is completely degenerate) has an effective specificity of 5 nucleotides.

A panel is a set of k-mer probes with the property that any given k long target is hybridized by one and only one probe in the panel. Thus, a panel is a complete and non-redundant set of probes.

The complexity C of a probe panel is the number of probes in the panel.

The sensitivity of a position within a panel is the set of different targets it can discriminate at that position. For example, a panel where the probes are either GC mixed or AT mixed at a position (denoted GC/AT) is sensitive to G-A, C-A, C-T and G-T differences (i.e. transitions), but not to transversions (G to C etc).

When probing with a full panel of probes, each position in the target is guaranteed to be probed by each position in the panel, i.e. by k staggered overlapping probes. However, the sensitivity of each position may be different, so that some differences in the target are only detectable by less than k probes.

For example, the panel given by (GCAT) (GC/AT) (GC/AT) (G/C/A/T) (G/C/A/T) (GC/AT) (GC/AT) (GCAT) has 8 positions (i.e. k=8). The first and last position are completely degenerate, so no change in the target is detected by those positions. Transitions (GC←AT) are detected by 6 positions, while transversions (GA←CT) are detected by only two positions in each probe. The effective specificity can be calculated by summing the effective specificity of each position: 0+0.5+0.5+1+1+0.5+0.5+0=4 bp.

For non-trivial targets, it will often be the case that probes are repeated in the target. Such probes lose their sensitivity to changes at any single position, since they will still hybridize to the other.

Given the length L of the target, one can calculate the probability (for each position in the target) that there is at least one probe sensitive to a change at that position. First, determine how many probes are sensitive to the change of interest in a repeat-free target. Call this k_{c}; k_{c} is 6 for transitions and 2 for transversions in the previous example.

The probability p(R) that any given probe is present in one or more of the other positions in the target (i.e. that it is repeated) is:

$$P ( R ) = 1 - ( \frac{C - 1}{C} ) \text{?}$$

$$\text{?} \text{indicates text missing or illegible when filed}$$

The probability p(S) that not all of the 2k_{c} sensitive probes are repeated is then:

P(S)=1−P(R)^{2k}^{_{c}}

The exponent is 2k_{c} because any change causes the disappearance of k_{c} probes and the appearance of k_{c} new probes.

The sensitivity given the target length may be calculated. For example, C=256, k_{c}=2, L=120 gives p=98%, i.e. the panel with 256 probes is sensitive to 98% of all transversions (and 100% of transitions, k_{c}=6). If only half of the probes in the panel are used, so that the effective k_{c}=1, then p=86% for transversions and 99.7% for transitions (k_{c}=3). The overall average sensitivity in a species like the human (which has 63% transitions) would be 95%.

The theory is strictly valid as long as the number of SNPs is low compared with the target length—i.e. as long as multiple SNPs do not occur within the length one probe. In practical experiments this is almost always true: for example, human genomic DNA contains about 1 SNP per 1000 nucleotides, and two SNPs within 7 bases is thus very unlikely.

In practice, at least two sensitive probes may be required to score a SNP (i.e. because hybridization data is error-prone). In that case, the probability P(S) becomes 1−p(R)₂^{kc-1} and the calculations are again straightforward.

When working with subsets of panels (in order to save time and reagents), it may desirable to nevertheless guarantee that any position in the target is probed on one strand or the other. In other words, a subset of probes is determined such that any k-mer that is not probed is guaranteed to be probed on the opposite strand. Such subsets can be obtained by placing (G/A), (C/T), (G/T) or (C/A) in the middle position. For example (G/A) will fail to probe G and A in the target, in which case the opposite strand is guaranteed to be either C or T, which are probed. Other variations are possible.

The (GC/AT) degenerate position has two desirable features. First, it guarantees that the individual oligos in each probe have similar melting point (since they will either be all GC or all AT). Second, the position will be sensitive to transitions which represent 63% of all SNPs in humans.

### Hybridization of Short Oligomer Probes

In the present invention, it is envisaged that a panel of probes is sequentially hybridized to the targets. In order to limit the complexity of the panel of probes, it is desirable to keep the probes short, preferably to have only 3-6 bp effective specificity.

The probes are stabilized in order for them to hybridize effectively, or at all. In addition, stabilization may help the probe compete with any internal secondary structure that may be present in the target. Stabilization can be achieved in many different ways.
Through stabilizing additives in the hybridization reaction, for instance salt, CTAB, magnesium, stabilizing proteins.
Through the addition of degenerate positions that extend the length of the probe without increasing its complexity. For example, a 6-mer probe extended with an ‘N’ positition would really be a mixture of four oligonucleotides, each 7 bases long. A (GC/AT) position—indicating a mix of G and C or a mix of A and T—would extend the probe by one base while only doubling the complexity (instead of quadrupling it).
Through modification of the probe chemistry, for example by means of locked nucleic acid (Exiqon, Denmark), peptide nucleic acid and or minor groove binder (Epoch Biosciences, US).
A combination of the above, for example a degenerate probe with LNA hybridized in CTAB buffer.
   Of these, the first will also stabilize the target (thus potentially inducing stable secondary structures which prevent hybridization). Methods that stabilize the probe selectively are preferred.

### Detecting Hybridization

Many approaches are known for detecting hybridization.
Direct fluorescence. The probe is labeled and hybridization is detected by the increased local concentration of probes hybridized to the target. This may require high magnification, confocal optics or total internal reflection excitation (TIRF).
Energy transfer. The probe is labeled with a quencher or donor and the target is labeled with counterpart donor or quencher. Hybridization is detected by the decrease of donor fluorescence and/or the increase in quencher fluorescence.
Single-base extension. The hybridized probe serves as primer for a single base extension reaction incorporating fluorescent dye (alternatively, released PPi maybe detected as in Pyrosequencing).

In one embodiment, the probe is labeled by a fluorophor detectable in an epifluorescence microscope or a laser scanner, for example Cy3. Many other suitable dyes are commercially available. The probe is hybridized to the array at a concentration optimized to permit detection of the local increase in concentration at a hybridized array feature, over the background present in all the liquid. For example, 400 nM may be used, or the probe may be hybridized at 1 nM up to 500 nM or even 500 nM up to 5 μM depending on the optical setup. The advantage of this detection scheme is that it avoids a washing step, so that detection can proceed at equilibrium hybridization conditions, which facilitates match/mismatch discrimination.

An energy transfer approach is described below.

The target carries a permanently hybridized helper oligonucleotide with a fluorescence donor. The helper is designed to withstand washes that would melt away the short probes. The probes carry a dark quencher. For example, the donor may be fluorescein and the quencher Eclipse Dark Quencher (Epoch Biosciences). Many other donor/quencher pairs are known (see e.g. Haugland, R. P., ‘Handbook of fluorescent probes and research chemicals’, Molecular Probes Inc., USA). In general, it is desirable to have a probe with a long Föbrster radius, capable of quenching over long distances. Hybridization is detected by the quenching of the donor fluorophor upon hybridization of the probe.

### Spectral Search and Alignment

Given the spectrum of a target, the location of the target within the reference sequence is sought, allowing for sequence differences. The search can be performed by simply scanning the reference sequence with a window of the same size as the target, computing an expected spectrum for each position and comparing the expected spectrum with the observed spectrum at the position. The highest-scoring position or positions are returned. Because the method of the invention generates very large numbers of hybridization spectra in a short time, it is important to. optimize the search step. For example, in a current implementation, spectral search proceeds at 1.2 billion matches per second on a high-end workstation, and we estimate that ten workstations will be required to keep up with a single sequencing instrument. It is another aspect of the invention to accelerate the search using programmable hardware, i.e. field-programmable gate arrays (FPGA). By translating the search algorithm to Mitrion-C (Mitrion AB, Sweden), an acceleration of 30 times can be achieved using just two FPGA chips in a single workstation computer.

Once one or more likely locations have been found, a modification to the reference sequence is sought that will explain any discrepancies between the observed and expected spectra. We may at this stage introduce relevant modifications to the reference sequence, e.g. SNPs, short indels, long indels, microsatellites, splice variants etc. For each modification or combination of modifications, we again compute a score for the similarity between the observed and expected spectra. The most likely modified reference sequence or sequences are returned. Methods for searching very large parameter spaces are known in the art, e.g. Gibbs sampling, Markov-chain Monte Carlo (MCMC) and the Metropolis-Hastings algorithm.

When comparing spectra, a simple binary overlap score may be used (scoring 1 for each probe that either does or does not hybridize in both spectra, 0 otherwise), or a more sophisticated statistical approach may use gradual or probabilistic measures of spectral overlap. Where multiple targets locate to the same position in the target, higher-level analysis may then be performed to assess the confidence in any sequence differences.

### An Apparatus for Automated High-Throughput Sequencing

Methods according to the present invention are particularly suitable for automation, since they can be performed simply by cycling a number of reagent solutions through a reaction chamber placed on or in a detector, optionally with thermal control.

In one example, the detector is a CCD imager, which may for example be operating by white light directed through a filter cube to create separate excitation and emission light paths suitable for a fluorophore bound to each target. For instance, a Kodak KAF-16801E CCD may be used; it has 16.7 million pixels, and an imaging time of ˜2 seconds. Daily sequencing throughput on such an instrument would be up to 10 Gbp.

The reaction chamber provides:
easy access for the optics.
a closed reaction chamber.
an inlet for injecting and removing reagents from the reaction chamber.
an outlet to allow air and reagents to enter and exit the chamber.

A reaction chamber may be constructed in standard microarray slide format as shown in FIG. 3, suitable for being inserted in an imaging instrument. The reaction chamber can be inserted into the instrument and remain there during the entire sequencing reaction. A pump and reagent flasks supply reagents according to a fixed protocol and a computer controls both the pump and the scanner, alternating between reaction and scanning. Optionally, the reaction chamber may be temperature-controlled. Also optionally, the reaction chamber may be placed on a positioning stage to permit imaging of multiple locations on the chamber.

A dispenser unit may be connected to a motorized valve to direct the flow of reagents, the whole system being run under the control of a computer. An integrated system would consist of the scanner, the dispenser, the valves and reservoirs and the controlling computer.

In accordance with a further aspect of the invention there is provided an instrument for performing a method of the invention, the instrument comprising: an imaging component able to detect an incorporated or released label, a reaction chamber for holding one or more attached templates such that they are accessible to the imaging component at least once per cycle, a reagent distribution system for providing reagents to the reaction chamber.

The reaction chamber may provide, and the imaging component may be able to resolve, attached templates at a density of at least 100/cm², optionally at least 1000/cm², at least 10 000/cm² or at least 100 000/cm², or at least 1 000 000/cm², at least 10 000 000/cm² or at least 100 000 000 per cm².

The imaging component may for example employ a system or device selected from the group consisting of photomultiplier tubes, photodiodes, charge-coupled devices, CMOS imaging chips, near-field scanning microscopes, far-field confocal microscopes, wide-field epi-illumination microscopes and total internal reflection miscroscopes.

The imaging component may detect fluorescent labels.

The imaging component may detect laser-induced fluorescence.

In one embodiment of an instrument according to the present invention, the reaction chamber is a closed structure comprising a transparent surface, a lid, and ports for attaching the reaction chamber to the reagent distribution system, the transparent surface holds template molecules on its inner surface and the imaging component is able to image through the transparent surface.

A further aspect of the invention provides a random array of single-stranded DNA molecules, wherein each said molecule consists of at least two tandem-repeated copies of an initial seguence, each said molecule is immobilized on a surface at random locations with a density of a density of between 10³ and 10⁷ per cm², preferably between 10⁴ and 10⁵ per cm², or preferably between 10⁵ per cm² and 10⁷ per cm², each said initial sequence represents a random fragment from an initial target DNA or RNA library comprising a mixture of single- or double-stranded RNA or DNA molecules, said initial sequences of all said DNA molecules have approximately the same length.

Generally, the molecules will comprise at least 100 tandem-repeated copies of an initial sequence, usually at least 1000, or at least 2000, preferably up to 20 000. The molecules may comprise 50 or more tandem-repeated copies of an initial sequence, which is detectable using standard microscopy.

Preferably, the initial sequences have the same length within 50% CV, preferably 5-50% CV, preferably within 10% CV, preferably within 5% CV i.e. such that the distribution is such that the coefficent of variation (CV) is e.g. 5%. CV=standard deviation divided by the mean. The initial sequences may have the same length.

The initial target library may for example be or comprise one or more of an RNA library, an mRNA library, a cDNA library, a genomic DNA library, a plasmid DNA library or a library of DNA molecules.

A further aspect of the invention provides a set or panel of probes wherein each probe consists of one or more oligonucleotides, each said oligonucleotide is stabilized, each said oligonucleotide carries a reporter moiety, the effective specificity of each probe is between 3 and 10 bp, the set of probes statistically hybridizes to at least 10% of all positions in a target sequence.

The effective specificity may be between 4 and 6 bp. The effective specificity may be 3, 4, 5, 6, 7 8, 9 or 10 bp.

The set of probes may statistically hybridize to at least 25%, at least 50%, at least 90% of all positions in a target sequence, or to 100% of all positions in a target sequence.

The set of probes may hybridize to 100% of all positions in a target sequence or its reverse complement, such that each position in the target or the reverse complement of the target at that position is hybridized by at least one probe in the set.

The target sequence may be an arbitrary target sequence.

A set of probes according to the invention may be stabilised by one or more of introduction of degenerate positions, introduction of locked nucleic acid monomers, introduction of peptide nucleic acid monomers and introduction of a minor groove binder.

The reporter moiety may for example be selected from the group consisting of a fluorophor, a quencher, a dark quencher, a redox label, and a chemically reactive group which can be labeled by enzymatic or chemical means, for example a free 3′-OH for primer extension with labeled nucleotides or an amine for chemical labeling after hybridization.

### Examples of Applications

### Gene Expression Profiling

By sequencing cDNA fragments at random, the expression level of the corresponding RNA can be quantified by counting the number of occurrences of fragments from each RNA. Structural features (splice variants, 5′/3′ UTR variants etc.) and genetic polymorphisms can be simultaneously discovered.

### Genetic Profiling

Shotgun sequencing of whole genomes can be used to genotype individuals by noticing the occurrence of sequence differences with respect to the reference genome. For example, SNPs and indels (insertion/deletion) can easily be discovered and genotyped in this way. In order to discriminate heterozygotic sites, dense fragment coverage may be required to ensure that both alleles will be sequenced.

Further aspects and embodiments of the present invention will be apparent to the skilled person in the light of the present disclosure. All documents cited anywhere in the specification are incorporated by reference.

### Example 1

### Preparing DNA Templates for Cantaloupe

### Input

Double stranded DNA template.

### Template Fractionation:

The restriction enzyme CviJ I* (EURx, Poland) was used, which recognizes 5′-GC-3′ and cuts blunt in between. The restriction reactions were prepared as follows:

| 1 ug Template | 1.5 ug Template | 2 ug Template |
|---|---|---|
| 2x reaction buffer | 2x reaction buffer | 2x reaction buffer |
| 25 ul | 25 ul | 25 ul |
| 0.3 units CviJ I* | 0.3 units CviJ I* | 0.3 units CviJ I* |
| Water to 50 ul | Water to 50 ul | Water to 50 ul |
| Total volume | Total volume | Total volume |
| 50 ul | 50 ul | 50 ul |

Reactions were incubated for 1 hour at 37° C.

The cleaved DNA was purified with PCR cleanup kit (Qiagen) according to manufacturer's protocol.

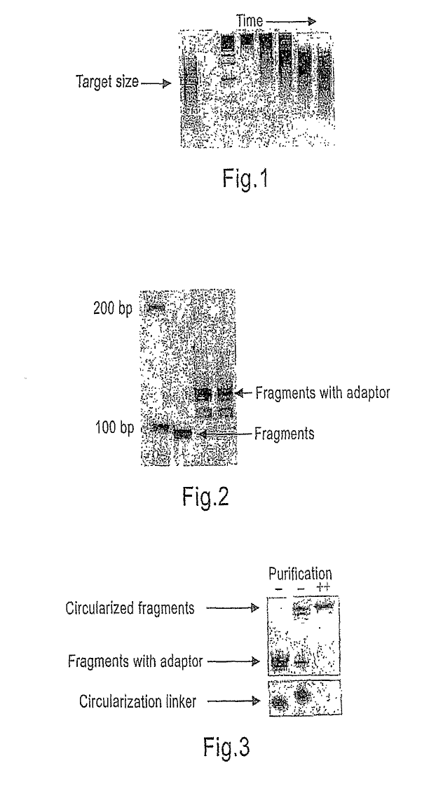

A fraction was analyzed on a 2% agarose gel to identify the optimal reaction conditions for the specific batch of template and enzyme (see FIG. 1, lanes 4-8).

The optimal cleavage reaction was repeated to get a total of 5 ug DNA (FIG. 1, lane 1).

### Template Size Selection:

The DNA was purified on an 8% non-denaturing PAGE (40 cm high, 1 mm thick). Each well was loaded with no more than 1 μg of DNA, and a 95-105 ladder was included, indicating the region of interest. The ladder consisted of 3 PCR fragments, at 95, 100 and 105 base pairs.

The gel was stained with SYBR gold, the results analyzed on a scanner, and the region of interest (95-105 bp) excised and electro-eluted with ElutaTube™ (Fermentas) according to manufactures protocol.

### Adaptor Ligation:

One adaptor was used for ligation.

| | |
|---|---|
| 5′ GCAGAATGCGCGGCCGCCTTAG 3′ | (SEQ ID NO: 11) |
| 3′ CGTCTTACGCGCCGGCGGAATC 5′ | |

It contained 5′ phosphates and an internal Not I site.

The following ligation mixture was prepared:

1 pmol of DNA (60-70 ng of fractionated sample)
25 pmol adaptor
Quick ligation buffer (NEB) 20-ul
Water up to 40 ul
Quick ligase (NEB) 2 ul
Total volume 42 ul

Incubated at 25° C. for 15 minutes. The reaction was purified using PCk cleanup (Qiagen) according to manufacturer's protocol. See FIG. 2.

### Restriction Digest Not I:

The following reaction was prepared:

Ligated DNA (all of it]
10x buffer (NEB) 10 ul
100x BSA 1 ul
Water up to 95 ul
Not I (50 units) 5 ul

Incubated at 37° C. for 4 hours or overnight. Samples were purified using PCR cleanup (Qiagen) according to manufactures protocol.

The purification was repeated with PCR cleanup to remove as much excess adaptors as possible.

### Circularization of Templates:

Single stranded circles were prepared by denaturing the samples in the presence of a linker oligonucleotide

(SEQ ID NO: 12)

5′-CGTCTTACGCGCCGGCGGAATCCGTCTTACGCGCCGGCGGAATC-
3′.

Specifically, the reaction was prepared as follows:

Ligated and Not I cut sample (everything)
5 pmol of linker oligo
Water up to 50 ul

Heated to 93° C. for 3 minutes, put on ice until cold, quick spin. 50 ul of 2× Quick ligation buffer (NEB) and 1 ul of Quick ligase (NEB) were added, mixed briefly, and Incubated 25° C. for 15 minutes.

At this stage the circles are formed and the samples can be used for RCA. See FIG. 3.

### Immobilization:

μM primer (identical to the circularization linker with an additional 5′-AAAAAAAAAA-C6-NH-3′ tail (SEQ ID NO:13), where C6 is a six-carbon linker and NH is an amine group) was immobilized on SAL-I slides (Asper Biotech, Estonia) in 100 mM carbonate buffer pH 9.0 with 15% DMSO, and incubated at 23° C. for 10 hours.

Remaining active sites on the slide surface were blocked by first soaking in 15 mM glutamic acid in carbonate buffer (as above, but 40 mM) at 30° C. for 40 minutes, then soaking in 2 mg/ml polyacrylic acid, pH 8.0 in room temperature for 10 minutes.

Circular templates were annealed at 30° C. in buffer 1 (2×SSC, 0.1%SDS) for 2 hours, then washed in buffer 1 for 20 minutes, then washed in buffer 2 (2×SSC, 0.1% Tween) for 30 minutes, then rinsed in 0.1×SSC, then rinsed in 1.5 mM MgCl₂.

### Amplification:

Rolling-circle amplification was performed for 2 hours in Phi29 buffer, 1 mM dNTP, 0.05 mg/mL BSA and 0.16 μ/μL Phi29 enzyme (all from NEB, USA) at 30° C.

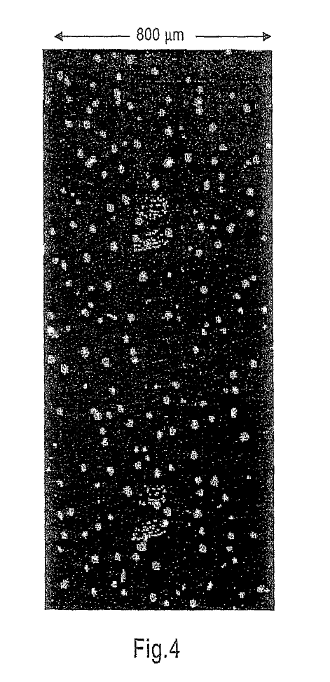

Reporter oligonucleotide complementary to the circularization linker and labeled with 6-FAM was annealed as above, followed by soaking in buffer 3 (5 mM Tris pH 8.0, 3.5 mM MgCl₂, 1.5 mM (NH₄J₂SO₄, 0.01 mM CTAB). FIG. 4 shows a small portion of a slide with individual RCA products clearly visible.

### Probe Panel Hybridization:

Each probe was designed according to the following scheme: (GCAT) (GC/AT) (GC/AT) (G/C/A/T) (GC/AT) (G/C/A/T) (GC/AT), each with locked nucleic acid (Exiqon, Denmark) at positions 2, 4 and 6 and with Eclipse dark quencher (Epoch Biosciences, USA) at the 3′ end.

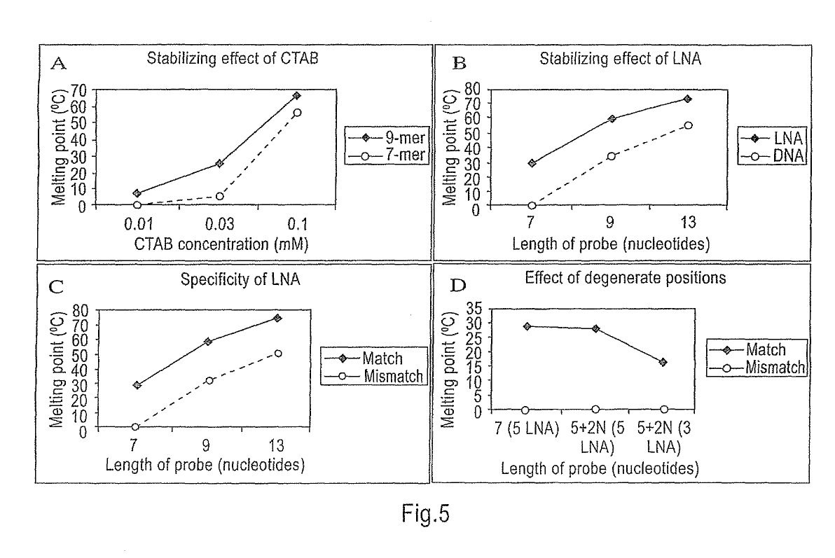

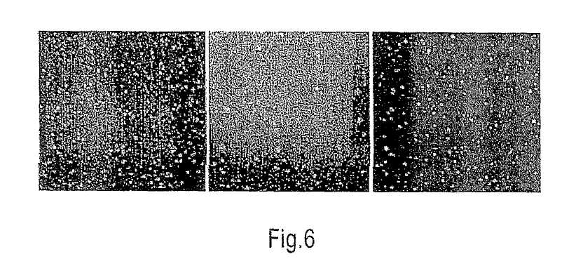

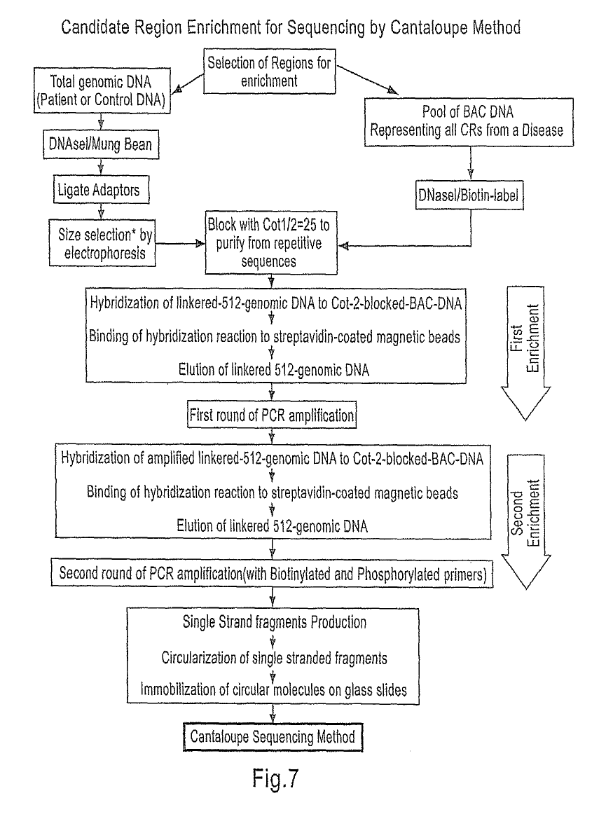

Probes were hybridized in buffer 3 at 100 nM. A temperature ramp was used for each probe to discover the optimal temperature for match/mismatch discrimination. FIG. 5 shows the result of hybridization of two match/mismatch pairs.

### Example 2

Preparation of Candidate Region Enrichment Fragments for Use with the Cantaloupe Sequencing Technology

### Step 1: Selection of Regions for Enrichment and Probe Preparation

In order to enrich a nucleic acid sample for candidate regions of interest, prior to sequencing with the Cantaloupe technology, the following exemplary protocol may be used.

The average candidate region size, based on genome wide association studies in diseases or complex genetic traits, such as Crohn's and psoriasis, is about half a megabase (0.5 Mb). All candidate regions associated with the disease can be selected, but in this example, 3 distinct regions from different chromosomes (region H: 453.5 kb, region R: 285.5 kb and region E: 193.6 kb) were selected, that together cover a total of 932.6 kb. In addition, in a separate example, only region E (193.6 kb) was selected to verify the effect of size on the enrichment method of the invention

A probe set in this method refers to specific DNA molecules that cover an entire chromosomal region, namely candidate regions resulting from Genizon GWS studies. The source of probes could be either YACs, BACs, cosmids or phages alone or in combination. In this example, BAC molecules are used.

Candidate regions are scanned for the availability of commercial BAC clones specific to the regions of interest and are ordered as the source material for probe preparation.

For probe preparation the following steps are performed:
a) BACs are stored at −80° C. in LB-Glycerol. With sterile pipette tips or an inoculating loop, the top of the vial is scraped.
b) The inoculum is then steaked on an LB agar plate (Chloramphenicol 12.5 μg/mL) to obtain single colonies.
c) The plate is then placed inverted at 37° C. overnight.
d) A single colony is selected from the freshly streaked selective plate and used to inoculate a starter culture of 5 ml LB (Chloramphenicol 12.5 μg/mL).
e) The culture is incubated for 8h with vigorous shaking (300 rpm) at 37° C.
f) A dilution is performed by taking 0.5-1.0 ml of the starter culture and adding it to 500 ml of selective LB medium (resulting in a 1/500 to 1/1000 dilution).
g) The diluted culture is then incubated at 37° C. for 12-16 h with vigorous shaking (˜300 rpm). A flask or vessel with a volume of at least 4 times the volume of the culture is preferably used. The culture should reach a cell density of approximately 3-4×10⁹ cells per ml.
h) From the 500ml overnight culture, the BAC-DNA is isolated using a QIAGEN® Large-Construct Kit as described by the manufacturer. Up to 150 μg of BAC-DNA free of bacterial genomic DNA is typically obtained.

### Step 2: Genomic DNA Preparation

DNA samples are selected from individuals affected by a particular disease (disease samples) or from unaffected individuals, which are used as controls (control samples). Disease samples represent specific combinations of haplotypes, including risk, neutral, protective and rare haplotypes, and cover all candidate regions of interest.

In this example, 3 different human genomic DNAs from healthy individuals were used. After standard preparation and purification of genomic DNA, the samples were treated consecutively by: bovine pancreatic DNase I and mung bean nuclease. The first enzymatic reaction was used to cause double strand breaks in the DNA in the presence of Mg²⁺, and the second enzymatic reaction produced blunt ended DNA fragments. The average fragment length (˜200 bp) and genomic DNA concentration were estimated by gel electrophoresis. The resulting fragments were then ready for adaptor ligation. The two different adaptors used in this example are described below and have no base modifications present in their sequence:

### Adaptor-1

(SEQ ID NO: 1)

5′-GCAGAATCCGAGGCCGCCT-3′ oligo name: UA-ADP1-512

(SEQ ID NO: 2)

5′-GACAAGGCGGCCTCGGATTCTGC-3′ oligo name: LA-ADP1-
512

### Adaptor-2

(SEQ ID NO: 3)

5′-AGTGGCGTGTCTTGGATGC-3′ oligo name: UA-ADP2-512

(SEQ ID NO: 4)

5′-CGATAACGCATCCAAGACACGCCACT-3′ oligo name:
LA-ADP2-512

The adaptors were designed to only ligate at the blunt end of the genomic DNA fragments.

a) The two adaptors were mixed and added to the ligation reaction in 75 fold excess (37.5 times each) in relation to the template genomic DNA fragments.

b) After the ligation reaction, the two strands were melted (72° C.) and Phusion polymerase (NEB, proofreading polymerase) was used to create blunt and double stranded ends.

c) The fragments ligated to the adaptors were then separated by electrophoresis on 3.5% Metaphor agarose (Cambrex, Baltimore, Md.). The region of interest was excised (fragment target size was ranging from 200 bp to 400 bp) and the DNA was purified using a GFX colunm (GE Healthcare).

d) The resulting purified genomic DNA fragments with adaptors (linkered-512 genomic DNA) were quantified by picogreen dye and adjusted to a 200 ng/ul concentration.

The resulting linkered-512 genomic DNA was concentrated by ethanol precipitation and kept for Step 4 (enrichment step).

### Step 3: BAC-DNA Probe Preparation

The BAC-DNA from step 1 was fragmented by DnaseI and biotinylated using a Biotin-Nick translation reaction mix (Roche) using 40 uM Biotin-16-dUTP. An isotope was included in the Nick translation reaction as a tracer to confirm that the biotinylation reaction had proceeded efficiently and to confirm binding of the BAC-DNA to the streptavidin-coated magnetic beads.

As described for the genomic DNA in step 2, repetitive sequences in the BAC-DNA were removed by blocking with Cot-1 DNA (Invitrogen) resulting in Cot-1-blocked-BAC-DNA, which was kept for Step 4 (enrichment step).

### Step 4: Enrichment Step

This step comprises two rounds of enrichment. Briefly, the first round enriches target DNA fragments from whole genomic DNA, while the second round enriches for target DNA fragments from the first round by reducing the amount of contaminating fragments. In both enrichment steps, the end products were DNA fragments of ˜250 bp. To quantify this enrichment, the resulting fragments were cloned into plasmids and transformed into bacteria. The resulting bacteria were streaked on appropriate LB plates. Independent clones were picked at random and probed for sequences specific to enriched regions. The formula used to calculate enrichment was:

Size HG/Size CR X % Specific sequence=Level of enrichment
Size HG: size of human genome (kb)
Size CR: size of the candidate region of interest (kb)
% SS: % of sequence specific to enriched region
   The table below summarizes the enrichment determination performed in this example:

| | | | | | | Percentage | |
|---|---|---|---|---|---|---|---|
| | | | | | | of | |
| | | | Good | | Sequences | sequences | |
| | | | sequences | | specific to | specific to | |
| | Size of | Clones | on | Successful | enriched | enriched | Level of |
| Experiment | region | sequenced | Megabace | blast | region | region | enrichment |
| A | 193.6 kb | 96 | 90 | 65 | 26 | 40% | 6200X |
| B | 932.6 kb | 96 | 81 | 44 | 15 | 34% | 1100X |

In experiment B, the conclusion is that 1 in 3 clones will have the target sequence from one of the 3 CR and the features (linkers) necessary for sequencing with the Cantaloupe technology.

### First Enrichment

Hybridization of linkered-512-genomic DNA (from step 2) to Cot-2-blocked-BAC-DNA (from step 3).

The linkered 512-genomic DNA (1 ug) was transferred to a 200 ul PCR tube and overlaid with mineral oil.

The sample was denatured by heating at 95° C. for 5 min and incubated at 65° C. for 15 min.

Cot-1-blocked BAC-DNA was added and the hybridization reaction was performed at 65° C. for 70 hours.

### Binding of the Hybridization Reaction to Streptavidin-Coated Magnetic Beads

The hybridization mixture was then added to streptavidin-coated magnetic beads (100 ul) at 15-25° C. for 30 min.

The beads were removed using a magnetic separator and the supernatant was discarded.

The beads were washed at room temperature for 15 minutes in 1 ml of 1×SSC, 0.1% SDS.

The beads were washed 3 times, each at 65° C. for 15 minutes in 1 ml of 0.1×SSC, 0.1% SDS.

The hybridized Tinkered 512-genomic DNA-Cot-1-blocked BAC-DNA was eluted from the magnetic beads by the addition of 100 ul of 0.1M NaOH and incubated at room temperature for 10 minutes.

The beads were removed using a magnetic separator. The beads contained the Cot-1-blocked BAC-DNA which was biotinylated and remained on the magnetic beads. The supernatant was neutralized with an equal volume of 1M Tris pH8, and then desalted with Centricon YM-30 columns (Millipore).

The resulting DNA (Tinkered 512-genomic DNA) was used as template for the first enrichment and amplification step described below.

### First Round of Amplification

The amplification reaction contains the Template DNA (Tinkered 512-genomic DNA) from above.

The primers used (10 uM each) were:

(SEQ ID NO: 5)

Forward: 5′-GACAAGGCGGCCTCGGATTCTG-3′

(SEQ ID NO: 6)

Reverse: 5′-CGATAACGCATCCAAGACACGC-3′

The other reagents used:
25 mM each dNTPs
5× Phusion reaction Buffer
Phusion Polymerase 1U
Water up to 50 ul

The amplification program was one denaturing cycle at 98° C. (30 sec) followed by 30 cycles of: 10 seconds denaturation at 98° C., 10 seconds of annealing at the primer melting temperature and 20 sec elongation at 72° C.

The amplification products were purified using a QIAquick PCR purification kit (QIAGEN) and kept as input DNA for a second enrichment step.

### Second Enrichment

The second enrichment was performed as described in the first enrichment step with the input DNA being the amplification products from the first enrichment. The second amplification was similar to the first amplification, described in the first enrichment above, with the difference being in the primers used (primers were identical in sequence but with modifications on the 5′-end):

(SEQ ID NO: 7)

Forward: 5′-BIOTIN-GACAAGGCGGCCTCGGATTCTG-3′

(SEQ ID NO: 8)

Reverse: 5′-PHO-CGATAACGCATCCAAGACACGC-3′

These modifications (biotinylation and phosphorylation) in the primers where included so as to ensure that the resulting DNA fragment were ready for the preparation (circularization) of input DNA for the sequencing technology “CANTALOUPE”.

### Example 3

### Preparing Data DNA Templates for Sequencing by Cantaloupe

### Step 1: Single Strand Production and Circularization

The purpose of this step is to retain only the phosphorylated single strand of the input double stranded target DNA generated in the second amplification step described in EXAMPLE 2.

The Dynabeads retained the input double stranded biotinylated and phosphorylated fragments. Incubation with 0.1M NaOH facilitated the release and isolation of the single stranded fragments of DNA containing the 5′-phosphate group necessary for the circularization step. The biotinylated strand is retained on the Dynabeads and the complementary strand is released in solution and used as input for the circularization step.

We formed single stranded circular molecules (necessary for use with the Cantaloupe sequencing technology) by denaturing the samples in the presence of the following biotinylated linker oligonucleotide:

(SEQ ID NO: 9)

5′-BIOTIN-CGTCTTACGCGCCGGCGGAATCCGTCTTACGCGCCGGCGG
AATC-3′

The reaction mixture consisted of: Single stranded linear fragments produced in step a (0.3 uM), 0.6 uM of the linker described above, and water up to 50 ul. The reaction mixture was heated to 65° C. for 2 minutes, and then cooled down to room temperature (the step took ˜15 minutes). Ice cold ligation mix (DNA ligase, 5U in 1× ligation buffer, Fermentas) was then added to the reaction mixture. The purpose of the addition of the ligase was to join the 3′ and 5′ ends of the single stranded fragments to permit the formation of circular molecules. For purposes of clarity, the circular molecules were hybridized to the biotinylated linkers to permit the juxtaposition of the 3′ and 5′ ends of the single stranded fragments. The biotinylated linkers were removed subsequently to obtain purified circular molecules, which were the input template DNA used for the Cantaloupe sequencing technology.

### Step 2: Purification of Circularized Molecules

The circularized molecules (annealed to the biotinylated linker from step 2) were then added to Dynabeads.

The beads were washed and left to dry after the final wash (as described in the manufacturers instructions).

The circular molecules were eluted from the beads using 40 mM NaOH.

The molecules were quantified by real time PCR.

The pure circular molecules are the template used for the rolling circle amplification steps present in the Cantaloupe sequencing technology.

Step 3: Immobilization of Circularized Molecules on Glass Slides used for Sequencing by Cantaloupe

Asper Biotech Genorama™ SAL, 0.15 or 1 mm slides were used in accordance with the manufacturer's instructions for handling and storage.

### Immobilization

5 uM RCA primer (identical to the circularization linker with an additional 5′-AAAPAAAAAA-C6-NH-3′ tail (SEQ ID NO:13), where C6 is a six-carbon linker and NH is an amine group) was immobilized on SAL-1 slides (Asper Biotech; see oligo used in Diagram A: 5′ XAAAAAAAAAAGCGTGTCTTGGATGCGTTATCG 3′ (SEQ ID NO:10) RCA-G-RING X═NH2-(CH2)6-PO4-Oligo) in 100 mM carbonate buffer pH 9.0 with 15% DMSO.

Samples were incubated at 30° C. for 1 hours.

The remaining active sites on the slide surface were blocked by first soaking in 15 mM glutamic acid in carbonate buffer (as above, but 40 mM) at 30° C. for 40 minutes, and then soaking in 2 mg/ml polyacrylic acid, pH 8.0 in room temperature for 10 minutes.

Circular templates were annealed at 30° C. in buffer 1 (2×SSC, 0.1% SDS) for 2 hours, then washed in buffer 1 for 20 minutes, then washed in buffer 2 (2×SSC, 0.1% Tween) for 30 minutes, then rinsed in 0.1×SSC, then rinsed in 1.5 mM MgCl₂.

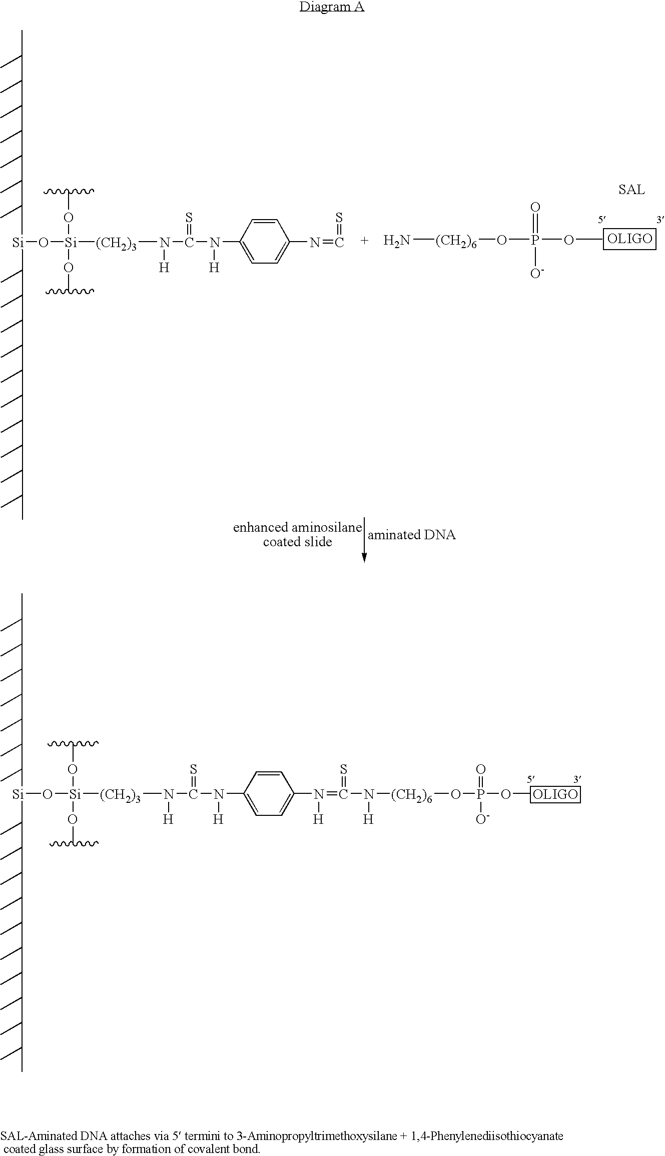

1. A nucleic acid sequencing method comprising:
enriching a nucleic acid sample for target sequences, wherein the nucleic acid sample is enriched through at least a first round of hybridization selection and amplification, and a second round of hybridization selection and amplification; and
sequencing said target sequences by shotgun sequencing by hybridization (SBH) of immobilized rolling circle amplicons.

2. The method of claim 1, wherein said DNA sample comprises genomic DNA.

3. The method of claim 2, wherein the DNA sample is prepared for enrichment by: fragmenting the DNA sample to create a population of DNA fragments; and ligating DNA adapters to said DNA fragments, wherein said DNA adapters contain primer binding sites.

4. The method of claim 3, wherein DNA fragments of about 500 base pairs or smaller are selected from the population of DNA fragments.

5. The method of claim 4, wherein DNA fragments of about 250 base pairs are selected from the population of DNA fragments.

6. The method of claim 3, wherein the fragmenting produces blunt-ended DNA fragments.

7. The method of claim 6, wherein DNA adaptors are ligated to the blunt-ended DNA fragments, each DNA adaptor having a blunt end.

8. The method of claim 1, wherein said first and second rounds of hybridization selection comprise, hybridizing the DNA sample with a nucleic acid probe having a tag, and capturing hybridized DNA with a ligand for said tag.

9. The method of claim 8, wherein the tag is biotin and the ligand is streptavidin.

10. The method of claim 9, wherein the streptavidin is immobilized on magnetic beads.

11. The method of claim 3, wherein DNA selected by the first round of hybridization selection is subsequently amplified in the first round of amplification.

12. The method of claim 11, wherein said first round of amplification is performed using polymerase chain reaction (PCR).

13. The method of claim 12, wherein said PCR is performed with primers complementary to the primer binding sites of said DNA adapters.

14. The method of claim 11, wherein the amplified DNA from the first round of amplification is further enriched in said second round of hybridization selection.

15. The method of claim 14, wherein the DNA selected by said second round of hybridization selection are subsequently amplified in said second round of amplification.

16. The method of claim 15, wherein said second round of amplification is performed using polymerase chain reaction (PCR).

17. The method of claim 16, wherein said PCR is performed with primers complementary to the primer binding sites of said DNA adapters.

18. The method of claim 17, wherein said PCR uses a forward primer that is modified on the 5′ end with a tag, and a reverse primer that is phosphorylated on the 5′ end.

19. The method of claim 18, wherein the tag on the forward primer 5′ end is biotin.

20. The method of claim 18, wherein products of said second round of amplification are denatured to create single stranded DNA.

21. The method of claim 20, wherein single stranded DNA having the tag are captured and removed.

22. The method of claim 21, wherein the tag is biotin and single stranded DNA having the biotin tag is captured and removed with streptavidin coated beads.

23. The method of claim 21, further comprising, circularizing the single stranded DNA having a phosphorylated 5′ end.

24. The method of claim 23, wherein the 5′-phosphorylated single-stranded DNA is circularized by hybridizing the 5′ and 3′ ends to an oligonucleotide linker, thereby holding the 5′ and 3′ ends in close proximity; and ligating the 5′ and 3′ ends to circularize the single-stranded DNA.

25. The method of claim 24, wherein the oligonucleotide linker has a tag.

26. The method of claim 25, wherein the oligonucleotide linker tag is biotin, and the oligonucleotide linker is captured and removed using streptavidin coated beads following circularization of the single-stranded DNA.

27. The method of claim 24, wherein the circularized DNA is immobilized on a solid support.

28. The method of claim 27, wherein the circularized DNA is immobilized by hybridization to an immobilized oligonucleotide, said immobilized oligonucleotide being immobilized through an amine.

29. The method of claim 27, wherein the immobilized, circularized DNA is amplified by rolling circle amplification.

30. The method of claim 29, wherein the rolling circle amplification products are sequenced using SBH.

31. The method of claim 1, wherein the target sequences are determined from whole genome association studies in a disease cohort.

32. The method of claim 31, wherein the disease cohort contains DNA samples from patients having one or more of Crohn disease, psoriasis, baldness, longevity, schizophrenia, diabetes, diabetic Retinopathy, ADHD, Endometriosis, asthma, an autoimmune related disease, an inflammatory related disease, a respiratory related disease, a gastrointestinal related disease, a reproduction related disease, a women's health related disease, a dermatological related disease and/or an ophthalmologic related disease.

33. The method of claim 8, wherein the nucleic acid probe is prepared from a bacterial artificial chromosome (BAC).

34. The method of claim 8, wherein repetitive sequences are blocked prior to hybridization with competitive DNA.

35. The method of claim 1, wherein the step of sequencing by SBH of immobilized rolling circle amplicons, comprises:
preparing a plurality of circular single-stranded DNA template molecule, each template molecule comprising a primer annealing sequence and a target sequence;
forming a random array of immobilized and amplified circular DNA template molecules, by: contacting the template molecules with an amplification primer that anneals to the primer annealing sequence thereby forming annealed primer/template complexes, and amplifying the template molecules by rolling-circle amplification, wherein the rolling circle amplification products are immobilized on a solid support;
probing the rolling circle amplification products with a panel of probes under test conditions,
determining for each probe in the panel whether the probe hybridizes to the target sequence of the rolling circle amplification product, or not, under the test conditions, thereby obtaining a hybridization spectrum for the target sequence;
comparing each hybridization spectrum to an expected hybridization spectrum for a reference sequence(s) in a reference database to determine the sequence of the target sequence.

36. The method of claim 35 further comprising, determining a sequence difference between the target sequence and the reference sequence(s), wherein the difference is one or more of a single nucleotide polymorphism, insertion, deletion, alternative splicing, an alternative transcriptional start site, alternative polyadenylation, and a microsatellite.

37. The method of claim 35, wherein the panel of probes comprises a plurality of probes wherein: each probe is a stabilized oligonucleotide carrying a reporter moiety, and the effective specificity of each probe is from 3 to 10 bp, wherein the panel of probes is such that at least 10% of all positions in the target sequence statistically hybridize with at least one probe in the panel.

38. The method of claim 37, wherein the effective specificity of each probe is from 4 to 6 bp.

39. The method of claim 37, wherein the panel of probes is such that at least 25% of all positions in the target sequence statistically hybridize with at least one probe in the panel.

40. The method of claim 39, wherein the panel of probes is such that at least 50% of all positions in the target sequence statistically hybridize with at least one probe in the panel.

41. The method of claim 40, wherein the panel of probes is such that at least 90% of all positions in the target sequence statistically hybridize with at least one probe in the panel.

42. The method of claim 41, wherein the panel of probes is such that at least 100% of all positions in the target sequence statistically hybridize with at least one probe in the panel.
